(19) United States
(12) Patent Application Publication
(10) Pub. No.: US 2024/0398492 A1
(43) Pub. Date: Dec. 5, 2024

(54) A ROBOTIC SURGICAL INSTRUMENT

(71) Applicant: CMR SURGICAL LIMITED, Cambridge, Cambridgeshire (GB)

(72) Inventors: David William Haydn Webster-Smith, Cambridge (GB); Ben Robert Chaplin, Cambridge (GB); Matthew David Knight, Sunnyvale, CA (US); Michal Piotrowski, Cambridge (GB)

(21) Appl. No.: 18/696,456

(22) PCT Filed: Sep. 30, 2022
(86) PCT No.: PCT/GB2022/052480

§ 371 Date: Mar. 28, 2024

(30) Foreign Application Priority Data
Oct. 1, 2021 (GB) 2114093.4

(51) Int. Cl. A61B 34/30; A61B 17/29; A61B 34/00

(52) U.S. Cl. CPC A61B 34/30; A61B 17/29; A61B 34/71; A61B 2034/302

(57) ABSTRACT

A robotic surgical instrument for performing a surgical procedure, the instrument comprising a shaft, an end effector comprising an electrical component and an articulation connecting the shaft to the end effector. The articulation permits the end effector to rotate relative to the shaft about a first axis. The robotic surgical instrument further comprises an electrical cable extending along the shaft. The electrical cable is configured to provide electrical current for the electrical component of the end effector. The robotic surgical instrument further comprises an electrical connector extending at least partially around a circumference surrounding the first axis. The electrical connector provides a sliding electrical connection between the electrical cable and the end effector, such that the end effector is permitted to rotate about the first axis independently of the electrical cable whilst the electrical connection between the electrical cable and the end effector is maintained.

### FIELD

This invention relates to a robotic surgical instrument that can be used for performing surgical procedures. For example, the robotic surgical instrument may be configured for performing electrosurgical operations.

### BACKGROUND

Electrosurgery is a term used to define surgical operations that are performed using instruments that are powered by a high frequency alternating electrical current. The alternating electrical current can be used to heat surgical tissue, thereby enhancing the functionality of the instrument, and reducing blood loss during dissection and resection. A common type of electrosurgery provides a radio frequency (RF) alternating current to an emitter that forms part of the electrosurgical instrument. RF waves emitted from the emitter heat the surgical tissue contacted by the instrument during cutting and/or sealing operations.

Electrosurgical instruments are commonly attached to surgical robots, as such robots are already connected to a source of alternating electrical current. Electrosurgical instruments that are attached to surgical robots typically comprise an end effector and one or more articulations, or joints, and the end effector may be expected to adopt a number of different rotational configurations as they are used to perform a surgical procedure. The end effector of an instrument is typically supplied with electrical current by one or more electrical cables connected thereto, the cables receiving current from an electrical power source connected to the surgical robot. The connection of the electrical cables to the end effector of the instrument means that, as the end effector of the surgical instrument is articulated, the connections and the electrical cables are moved with the end effector of the instrument to which they are connected. This movement of the cable can result in bending, wear and strain on the electrical cable, which could result in damage (e.g. a break) in the cable.

### SUMMARY

According to a first aspect, there is provided a robotic surgical instrument for performing a surgical procedure, the instrument comprising: a shaft; an end effector comprising an electrical component; an articulation connecting the shaft to the end effector, the articulation permitting the end effector to rotate relative to the shaft about a first axis; an electrical cable extending along the shaft and being configured to provide electrical current for the electrical component of the end effector; and an electrical connector extending at least partially around a circumference surrounding the first axis, the electrical connector providing a sliding electrical connection between the electrical cable and the end effector, such that the end effector is permitted to rotate about the first axis independently of the electrical cable whilst the electrical connection between the electrical cable and the end effector is maintained.

The electrical connector may extend around the entirety of the circumference surrounding the first axis.

The electrical connector may be in the shape of a ring.

The electrical cable may be connected to the electrical connector at a section of the electrical connector that is closest to the shaft.

The electrical connector may be integral with the electrical cable.

The robotic surgical may further comprise an insulating component configured to house the electrical connector within the surgical instrument.

The articulation may comprise a supporting body connected to the end effector by a first joint and to the shaft by a second joint, the first joint permitting the end effector to rotate relative to the shaft about the first axis.

The insulating component may be configured to interfere with the supporting body such that rotation of the insulating component about the first axis is limited when the end effector rotates about the first axis.

The insulating component may be secured at a proximal end to the supporting body.

The electrical connector may be electrically connected to the end effector by connecting means.

The connecting means may be configured to apply a compressive force to the electrical connector to constrain the electrical connector against the insulating component.

The connecting means may be a disc spring.

The connecting means may comprise a securing mechanism configured to secure the connecting means to the end effector.

The securing mechanism may be a keyed joint.

The insulating component may comprise an outer surface that faces an end effector element of the end effector and a groove that is recessed from the outer surface and configured to house at least part of the electrical connector.

The insulating component may be a locking ring configured to be secured to the end effector, and the electrical connector may be configured to extend at least partially around the circumference of the locking ring.

The insulating component may comprise a cylindrical outer surface and a groove that is recessed from the outer surface and configured to house at least part of the electrical connector.

The electrical connector may comprise an outer surface that is arranged to provide the sliding electrical connection and that is provided with an anti-friction coating.

The instrument may be a monopolar surgical instrument or a bipolar surgical instrument.

The electrical cable may a first electrical cable, and the electrical connector may be a first electrical connector; the end effector may comprise a first end effector element configured to rotate about the first axis and a second end effector element configured to rotate about a second axis; the first electrical connector may provide a sliding electrical connection between the first electrical cable and the first end effector element of the end effector; and the robotic surgical instrument may further comprise a second electrical cable and a second electrical connector, the second electrical connector providing a sliding electrical connection between the second electrical cable and the second end effector element of the end effector, such that the second end effector element is permitted to rotate about the second axis independently of the second electrical cable whilst the electrical connection between the second electrical cable and the second end effector element is maintained.

The second electrical connector may extend at least partially around a circumference surrounding the second axis.

The first axis and the second axis may be collinear.

The insulating component may be located between the first end effector element and the second end effector element such that the first electrical connector is electrically isolated from the second electrical connector.

The first and second end effector elements may be a pair of blades of a gripping tool.

The first axis may be transverse to a longitudinal axis of the shaft.

The electrical component may be an electrode or an emitter of energy waves.

### BRIEF DESCRIPTION ON THE FIGURES

The present invention will now be described by way of example with reference to the accompanying drawings. In the drawings:

FIG. 1 illustrates a surgical robot;

FIGS. 2A and 2B illustrate the configuration of a surgical instrument;

FIG. 3 illustrates an improved configuration of the distal portion of a surgical instrument comprising a single end effector element;

FIG. 4 illustrates an improved configuration of the distal portion of a surgical instrument comprising two end effector elements;

FIG. 5 illustrates an isometric view of the insulating component of FIG. 4;

FIG. 6 illustrates a first alternative configuration of the distal portion of the surgical instrument illustrated in FIG. 4;

FIG. 7 illustrates a second alternative configuration of the distal portion of the surgical instrument illustrated in FIG. 4;

FIG. 8 illustrates a third alternative configuration of the distal portion of the surgical instrument illustrated in FIG. 4.

### DETAILED DESCRIPTION

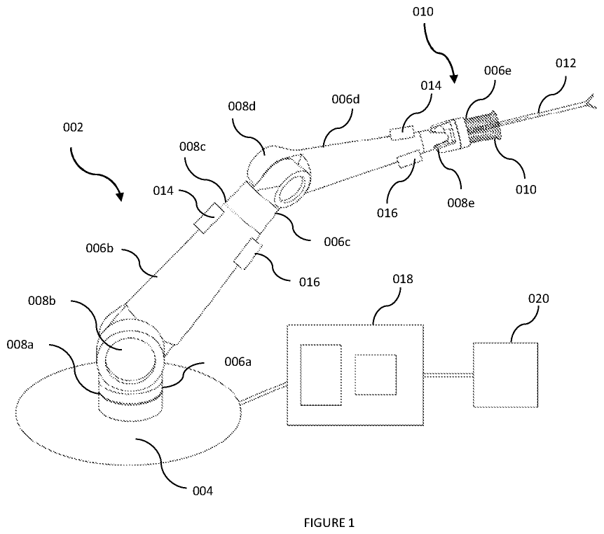

FIG. 1 illustrates a surgical robot having an arm 002 which extends from a base unit 004, and a plurality of rigid limbs 006a-e that are coupled together by a plurality of joints 008a-e. The joints 008a-e are configured to apply motion to the limbs 006a-e. Each joint of the arm has one or more drive sources 014 that can be operated to cause rotational motion at the respective joint. The most distal limb of the robot carries an attachment 010 for a surgical instrument 012. Each joint further comprises one or more sensors 016 that provide sensory information regarding the configuration at that joint. Controllers for the drive sources and sensors are distributed within the robot arm. The controllers are connected via a communication bus to a control unit 018 comprising executable software to control the operation of the drive sources and cause the arm to operate. The drive sources can be operated in dependence on inputs from the sensors and from a surgeon via a surgeon command interface 020.

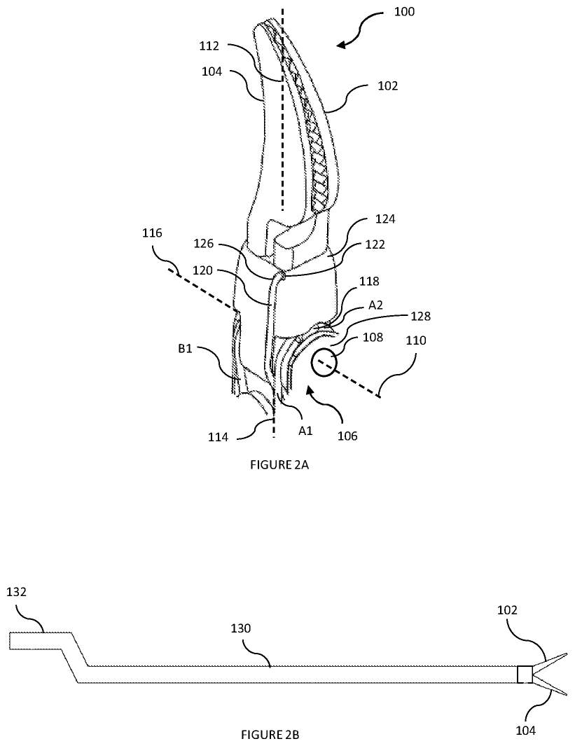

The surgical instrument comprises an end effector for performing an operation. The end effector may take any suitable form. For example, the end effector may be smooth jaws, serrated jaws, a gripper, a pair of sheers, a needle for suturing, a camera, a laser, a knife, a stapler, a cauteriser or a suctioner. The surgical instrument further comprises an instrument shaft and an articulation located between the instrument shaft and the end effector. The articulation comprises one or more joints that permit the end effector to move relative to the shaft of the instrument. The joints in the articulation are actuated by driving elements. These driving elements are secured at the other end of the instrument shaft to interface elements of an instrument interface (illustrated by reference numeral 132 in FIG. 2B). The driving elements are elongate elements that extend from the joints in the articulation through the shaft to the instrument interface 132. Each driving element can be flexed transverse to its longitudinal axis in the specified regions. In an example, the driving elements may be cables.

The surgical instrument may be an electrosurgical instrument. The term “electrosurgical instrument” within the context of this application is used to refer to an instrument that has an end effector with an electrical component to which electrical current is to be provided for the correct operation of the surgical instrument. For example, an electrosurgical instrument can perform electrosurgery, electrocautery, diathermy or any other form of surgical procedure involving the operation of an electrical component in the end effector, e.g. for heating of surgical tissue using electrical current. The end effector(s) of an electrosurgical instrument may be similar to those of surgical instruments that are not capable of performing electrosurgery. For example, the end effector of an electrosurgical instrument may be smooth jaws, serrated jaws, a pair of shears, a knife, or a cauteriser.

If the end effector comprises one or more electrically conducting components (e.g. metal components such as metal jaws or a metal blade) then these one or more electrically conducting components may be the “electrical component(s)” to which electrical current is to be provided. For example, the end effector of an electrosurgical instrument may comprise an electrical component configured to receive electrical current and to transform that current into an alternative type of energy that is used to generate heat. The alternative type of energy may be radio frequency (RF) energy, microwave energy, ultrasound energy or energy of another suitable wavelength that is able to heat surgical tissue. The electrical component may be an electrode or a type of emitter of energy waves. The type of wave emitted by the electrical component is dependent on the frequency of the electrical current that is provided to the end effector. As another example, the end effector of an electrosurgical instrument may comprise an electrical component configured to receive electrical current and to conduct that current so that it passes into the surgical tissue of the patient, wherein when the electrical current flows through the surgical tissue it generates heat. An end effector may comprise more than one type of electrical component. For example, an end effector may comprise both an emitter of microwaves and an electrode. The different types of electrical components may be used for different purposes. For example, a first type of electrical component may be used to cut surgical tissue and second type of electrical component may be used to seal this tissue. Where the end effector comprises more than one type of electrical component, the electrical cables required to supply electrical current to each electrical component may be of the same type or of different types, depending on the requirements for the respective types of energy emitted by the components.

The instrument is supplied with electrical current by an electrical cable that extends along (e.g. through) the shaft. The electrical cable is attached at its first end to the instrument and at its second end to a source of electrical current (e.g. alternating current), that may be part of and located at the base of the surgical robot, for example. In an alternative example, the source of electrical current may be a standalone unit that is separate to the surgical robot. The electrosurgical instrument further comprises an electrical connector that provides an electrical connection between the electrical cable and the electrical component of the end effector(s).

An example of the distal end of an electrosurgical instrument is illustrated in FIGS. 2A and 2B. The distal end of the instrument comprises an end effector 100. The end effector 100 has a pair of end effector elements 102, 104. The end effector 100 is connected to the distal end of the shaft 130 of the instrument by an articulation 106. The shaft 130 is connected at its proximal end to an interface for attaching to a robot arm. Articulation 106 comprises joints that permit movement of the end effector 100 relative to the shaft 130. A first joint 108 permits the first end effector element 102 to rotate about a first axis 110. The first axis 110 may be transverse to the longitudinal axis 114 of the shaft 130.

A second joint permits the second end effector element 104 to rotate about a second axis 116. The second axis may also be transverse to the longitudinal axis 114 of the shaft. The second axis may be parallel to the first axis 110. In the example illustrated in FIGS. 2A and 2B, the first and second axes 110, 116 are the same axis, i.e. they are collinear. This is illustrated in FIG. 2A. However, in alternative examples, the second axis is not the same as the first axis 110. For example, the second axis may be parallel to but offset from the first axis 110. The offset may be in a direction defined by (e.g. along) the longitudinal axis 114 of the shaft. The offset may be in a direction that is not defined with respect to (e.g. not along) the longitudinal axis 114 of the shaft.

The first end effector element 102 and the second end effector element 104 are independently rotatable about the first axis 110 and the second axis 116 respectively because of the first and second joints. The end effector elements may be rotated in the same direction or different directions by the first and second joints. The shaft 130 terminates at its distal end at a third joint (not shown). The third joint permits the end effector 100 to rotate about a third axis. The third axis may be transverse to the first axis 110.

The articulation 106 comprises a supporting body 128. At a first end, the supporting body 128 is connected to the end effector 100 by the first joint 108 and the second joint. At a second end opposing the first end, the supporting body 128 is connected to the shaft 130 by the third joint. The first joint 108 and second joint permit the end effector elements 102, 104 to rotate relative to the supporting body about the first and second axes 110, 116. The instrument illustrated in FIGS. 2A and 2B is in a straight configuration. In this configuration, the end effector 100 is aligned with the shaft 130 of the instrument. That is, the longitudinal axis 112 of the end effector is coincident with longitudinal axis 114 of the shaft.

Each joint of the instrument is driven by a pair of driving elements. This means that each joint of the instrument is independently driven. The first joint 108 is driven by a first pair of driving elements A1, A2. The second joint is driven by a second pair of driving elements B1, B2. FIGS. 2A and 2B illustrate only the first of this second pair of driving elements, B1, with B2 being routed around the second side of the second joint similarly to the routing of driving element A2 around a second side of the first joint 108 to the first driving element A1. At one point, driving elements of a pair of driving elements are secured to their corresponding joint. For example, the first pair of driving elements A1, A2 comprises a ball feature 118 that is secured to the first joint 108. This ensures that when the pair of driving elements is driven, the drive is transferred to motion of the joint about the first axis 110. A corresponding ball feature is secured to the second joint to ensure that drive is transferred to motion of the second joint about the second axis 116.

The end effector elements 102, 104 are illustrated in FIGS. 2A and 2B as a pair of opposing serrated jaws. However, the end effector elements may take any alternatively suitable form such as smooth jaws, a pair of shears or a pair of blades of a gripping tool. Whilst the end effector of the instrument illustrated in FIGS. 2A and 2B comprises two end effector elements, it is appreciated that in alternative examples the instrument may comprise a single end effector element, or more than two end effector elements.

The instrument further comprises at least one electrical cable 120. The electrical cable is configured to provide electrical current to the end effector. The electrical cable may be a flexible cable. A flexible cable is one that is resistant to breaking when it is bent. Alternatively, the electrical cable may be a rigid cable. A rigid cable is one that is more prone to breaking when it is bent. The electrical cable may be made from materials of any suitable stiffness. The electrical cable may comprise a single section. In other words, the electrical cable may be formed of continuous lengths of material along its longest dimension. Alternatively, the electrical cable may be formed of more than one section (or lengths of material). This latter example allows for the cable to have interchangeable sections, or parts, that pass through different parts of the surgical instrument. For example, the cable may have a first section that extends through the end effector of the instrument. The first section may be connected to a second section that extends through the shaft of the instrument (which interfaces with the robot arm). The cable may further comprise a third section that extends through the proximal housing of the instrument towards the robot arm. The different sections of the electrical cable may be physically connected together by any suitable means to form a continuous electrical connection. The different sections of the electrical cable may have different stiffnesses. For example, a first section of the electrical cable may be more flexible relative to a second section of the cable. By assigning different sections of the electrical cable different stiffnesses, the material properties of the cable can be matched to the amount of articulation that is experienced by each section of the cable by virtue of its location within the surgical instrument.

The electrical cable 120 is connected to the end effector by an electrical connector 122. The electrical connector 122 may plug directly into the end effector. The electrical connector 122 provides a fixed electrical connection between the electrical cable 120 and the end effector 100. That is, when connected to the end effector 100, the end effector 100 is not able to move independently of the electrical connector 122. Thus, as an end effector element 102,104 of the end effector rotates about its respective joint, the electrical connector 122 moves with that end effector element.

The end effector comprises at least one electrical component (e.g. the metal jaws of the end effector elements 102 and 104) configured to receive electrical current from the electrical cable 120 and for example transform that current into energy that is used to generate heat. Energy from the electrical component is transferred to a patient via the end effector element 102, 104. In one example, the electrical component is an electrode. Where the electrical component is an electrode, the electrode is configured to heat surgical tissue by ohmic heating when electrons pass through the tissue. Alternatively, if the electrical component is an emitter of energy waves, the waves are transferred to a part of the end effector element that is configured to contact the body of a patient. The waves are in turn configured heat and vaporise the water content of surgical tissue. The waves generated by the emitter may for example be radio frequency (RF) waves, microwaves, infrared waves, infrared waves, ultrasound waves or waves of any other suitable frequency that can be used to heat organic tissue during a surgical procedure. The type of wave emitted may be dependent on the frequency of current that is provided to the end effector. The instrument also comprises an external insulator 124 configured to insulate the electrically charged components of the instrument such as the electrical connector and the electrical component from other parts of the surgical instrument and from the patient.

Where the instrument comprises a single electrical component, the instrument is described as a monopolar instrument. For a monopolar instrument, the single electrical component of the electrosurgical instrument may be an electrode that is configured to contact the tissue of a patient and heat the tissue at a first location on the patient. The electrode of the monopolar instrument may be referred to as an “active” electrode. A second electrode may be external to the instrument and connected to a second location on the patient, and to disperse current from the active electrode from the patient. The second electrode may be referred to as a “dispersive” electrode. The monopolar instrument requires a single electrical cable to provide electrical current to its single electrical component. Thus, in turn, a monopolar instrument requires a single electrical connector to provide an electrical connection between its electrical cable and electrical component.

In an alternative example, the instrument may comprise a pair of electrical components. In this example, the electrosurgical instrument is described as a bipolar instrument. A first electrical component may be positioned on a first side of the end effector, and a second electrical component may be positioned on a second side of the end effector that opposes the first side. Where an end effector comprises two end effector elements, a first electrical component may be (or may be attached to) the first end effector element and a second electrical component may be (or may be attached to) the second end effector element. Where the end effector comprises two end effector elements, alternating energy waves may oscillate between the two end effector elements when the electrical components are charged, heating the intervening tissue by oscillation of intracellular ions.

A problem associated with the arrangement of the instrument illustrated in FIGS. 2A and 2B is that the fixed connection between the electrical connector 122 and the end effector 100 means that the electrical cable 120 is forced to move (e.g. bend or stretch) when the end effector is articulated. More specifically, when the first and/or second end effector elements 102, 104 are rotated about their respective first and second joints, the electrical cable will move with the elements and experience bending or stretching. The electrical cable 120 is also forced to bend when the end effector is rotated about the third joint. The electrical connector 122 may be orientated perpendicularly to the path of the electrical cable 120 along the shaft 130 of the instrument. This perpendicular orientation may cause a deflection 126 in the electrical cable that is particularly vulnerable to bending stresses and abrasion. Thus, the electrical cable 120 and the electrical connector 122 may be prone to damage due to rotation of the end effector elements. This damage may reduce the overall lifespan of the surgical instrument, as the electrical cables may break before the mechanical failure of other parts of the surgical instrument. The inventors of the present application have identified that there is a need for a more durable electrical connection that can increase the life span of electrosurgical instruments.

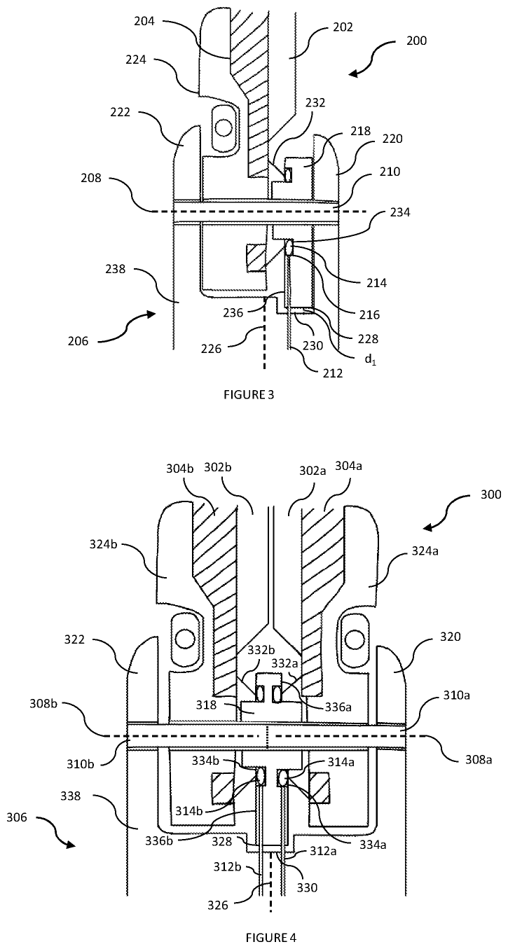

An example of an improved arrangement for an electrosurgical instrument to be attached to a surgical robot is illustrated by the cross-sectional view in FIG. 3. The electrosurgical instrument of FIG. 3 is a monopolar instrument comprising an end effector with a single end effector element.

As with the instrument illustrated in FIGS. 2A and 2B, the instrument of FIG. 3 comprises a shaft and an end effector 200. The end effector 200 comprises a single end effector element 202 and an electrical component 204. The electrical component 204 is part of (or attached to) the end effector element 202. The electrical component 204 is configured to receive electrical current. The electrical component 204 may be further configured to transform the received electrical current into energy that is used to generate heat. Energy from the electrical component 204 is transferred to a patient via the end effector element 202, e.g. via the electrical component 204. The instrument also comprises an external insulator 224 configured to insulate the electrically charged components of the instrument such as the electrical connector and the electrical component from other parts of the instrument and from the patient.

The instrument further comprises an articulation 206 connecting the shaft to the end effector 200. The articulation 206 permits the end effector element 202 to rotate relative to the shaft about a first axis 208. In particular, the articulation 206 comprises a first joint 210 about which the end effector element 202 is configured to rotate. In other words, the first joint 210 permits the end effector 200 to rotate relative to the shaft about the first axis 208. As with the instrument illustrated in FIGS. 2A and 2B, the articulation 206 comprises a supporting body 238. The supporting body 238 is rotatably connected to the end effector element 202 by the first joint 210. The supporting body 238 is also connected to the shaft by a second joint. The first axis 208 may be transverse to the longitudinal axis 226 of the shaft.

The supporting body 238 may comprise a first tine 220 and a second tine 222. The first and second tines extend from the base of the supporting body 238 towards the distal end of the instrument. The first tine 220 may oppose the second tine 222. That is, the first tine 220 may be located on an opposite side of the supporting body to the second tine 222. The first tine 220 and the second tine 222 may be spaced apart, such that at least a portion of the end effector element 202 is permitted to rotate between the tines. The first tine 220 and the second tine 222 may extend towards the distal end of the instrument 200 in a direction that is parallel to the longitudinal axis 226 of the shaft when the instrument is in a straight configuration.

The instrument further comprises an electrical cable 212. The electrical cable 212 may be composed as is described with respect to FIG. 1 above. The electrical cable 212 extends along the shaft of the instrument. The electrical cable 212 may extend through the shaft of the instrument. Alternatively, the electrical cable 212 may extend along an exterior surface of the shaft. The electrical cable 212 may extend along the shaft in a direction that is substantially parallel to the longitudinal 226 axis of the shaft, when the instrument is in a straight configuration. The electrical cable 212 is configured to provide electrical current to the electrical component 204 of the end effector 200. The electrical cable 212 may be a coaxial cable. Coaxial cables are advantageous in carrying high frequency electrical current for electrosurgical instruments due to the low losses that they provide. Losses would incur distortions, such as to the phase of electrical signals, which are problematic for electrosurgical instruments which need to transmit a specific frequency of energy wave phase in order to heat the water content of surgical tissue. The electrical cable 212 is electrically connected to the end effector 200 at a first end. More specifically, the electrical cable 212 is electrically connected to the electrical component 204 of the end effector at a first end. The electrical cable 212 is electrically connected at a second end to a source of electrical current. In this way, the electrical cable 212 can provide electrical current from the source of electrical current to the electrical component 204 of the end effector.

The instrument further comprises an electrical connector 214. The electrical connector 214 is configured to provide an electrical connection between the electrical cable 212 and the end effector 200. More specifically, the electrical connector 214 is configured to provide an electrical connection between the electrical cable 212 and the electrical component 204 of the end effector. The electrical connector 214 differs from the corresponding connector of FIGS. 2A and 2B in that it is configured to provide a sliding electrical connection between the electrical cable 212 and the end effector 200. That is, the electrical connector 214 is configured to transfer electrical current from the electrical cable 212 to the end effector 200, whilst being able to slide relative to the end effector. The term “sliding” referenced herein is used to describe the ability of the end effector 200 to move independently of the electrical connector, and vice versa, in at least one degree of freedom whilst maintaining an electrical connection. As described in more detail below, by providing a sliding electrical connection, the electrical connector 214 is electrically connected to the end effector but not physically fixed to a particular point on the end effector. The degree of freedom may be a translational degree of freedom. Alternatively, the degree of freedom may be a rotational degree of freedom. The sliding connection between the end effector 200 and the electrical connector 214 may act about the first axis 208. In other words, the sliding connection may provide the end effector 200 with a rotational degree of freedom about the first axis 208, independently of the electrical connector.

The sliding electrical connection means that the electrical connector 214 is not fixed with respect to the end effector 200. In particular, the electrical connector 214 may not be rotationally fixed with respect to the end effector 200 about the first axis 208. This means that the end effector 200 may be permitted to rotate independently of the electrical connector 214, and vice versa. At the same time, the electrical connection between the electrical cable 212 and the end effector 200 is maintained. That is, whilst the electrical connector 214 is free to rotate with respect to the end effector 200 about the first axis 208, the electrical connector 214 is still able to transfer electrical current from the electrical cable 212 to the electrical component 204 of the end effector 200. An electrical connection may be maintained by engagement of an outer surface of the electrical connector 214 against an inner surface 204 of the electrical component 204 of the end effector 200. The electrical connector 214 may be disposed between a tine of the supporting body 238 and the end effector 200 such that an electrical connection between the electrical component 204 and the electrical connector 214 can be maintained.

The electrical connector 214 may be substantially flat, or two-dimensional. That is, the electrical connector may have a depth that is significantly smaller in dimension than its width and its length. The surface area of the electrical connector 214 may form an enclosed area within an outer perimeter and an inner perimeter, with an open space between the inner perimeter and the centre of the connector. The electrical connector 214 may be connected to the end effector 200 around the circumference of the first joint 210. The electrical connector 214 may extend at least partially around a circumference surrounding the first axis 208. That is, the electrical connector 214 may be in the shape of an arc the extends around a circumference surrounding the first axis 208. An “arc” within this context refers to a part of the circumference of a circle, such as that which surrounds the first axis 208. An advantage associated with designing the electrical connector in this way is that the force that is exerted by the connector onto the end effector element is distributed around the first axis to the extent that the connector extends around the axis. This distribution of force around the first axis is useful in ensuring the correct alignment of the end effector element about that axis. The electrical connector 214 may therefore form a track around the first joint 210, and therefore around the first axis 208. For example, the outer perimeter of the electrical connector 214 may be in the shape of a semicircle that extends around the first axis 208. The outer perimeter of the electrical connector 214 may alternatively form the majority, or all, of a circle. The outer perimeter of the electrical connector 214 may have an elliptical shape. The electrical connector 214 may alternatively have any other shape that also maintains a sliding electrical connection as the end effector 200 is moved through its entire permitted range of motion about the first joint 210. The electrical connector 214 may extend around the entirety of the circumference surrounding the first axis 208. In this example, the electrical connector 214 may be in the shape of a circle. The electrical connector may alternatively be in the shape of an oval. The electrical connector 214 may be in any other suitable shape that is capable of providing a sliding connection. In one example, the electrical connector 214 may be in the shape of a ring, or a loop. The electrical connector 214 may be in the shape of a torus.

The electrical cable 212 may be connected to the electrical connector 214 at a section of the electrical connector that is closest to the shaft. In other words, the connection between the electrical cable 212 and the electrical connector 214 may be at a proximal end of the electrical connector. That is, the electrical cable 212 may connect to the electrical connector 214 at a lowermost part of the electrical connector, when the instrument is in the straight configuration. This lowermost part is illustrated in FIG. 3 by reference numeral 216. An advantage of the electrical cable 212 being connected to the lowermost part 216 of the electrical connector is that the electrical cable 212 can be directed down the shaft from the connector, in a direction parallel to the longitudinal axis 226 of the shaft. This means that bending and deflection of the electrical cable is minimised. However, in alternative examples the electrical cable 212 may connect to the electrical connector 214 at a different part of the electrical connector. For example, the electrical cable 212 may connect to the electrical connector 214 at a part of the electrical connector 214 that is 90 degrees to the lowermost part 216. The electrical cable 212 may be physically fixed to the electrical connector 214, so that the electrical cable 212 and the electrical connector 214 cannot move independently of one another.

In an example, the electrical connector 214 may be integral with the electrical cable 212. That is, the electrical connector 214 forms the same component as the electrical cable 212. The electrical connector 214 and the electrical cable 212 may be manufactured together, such that they cannot be separated from each other, prior to attachment of the connector and the cable to the instrument. Alternatively, the electrical connector 214 may be a separate component to the electrical cable 212. In this alternative example, the electrical connector 214 may be attached to the electrical cable 212 during manufacturing of the surgical instrument. Thus, the electrical connector 214 may be separated from the electrical cable 212. This may be considered to be advantageous as it enables the electrical cable 212 and/or the electrical connector 214 to be replaced as necessary during use of the instrument.

The instrument may further comprise an insulating component 218. The insulating component 218 is configured to house the electrical connector 214 within the surgical instrument. The insulating component 218 is also configured to prevent the dispersion of electrical current to locations other than the electrical component 204, such as the supporting body 238 of the articulation. In other words, the insulating component 218 is configured to electrically insulate the electrical connector 214 from components of the instrument that are not the electrical component 204. To provide electrical insulation, insulating component 218 may be made of an insulating material such as plastic. For example, the insulating component may be made of a plastic such as Teflon. The insulating component may be made of a plastic that is also suitable for pharmaceutical use, such as polyetheretherketone (PEEK). The insulating component located within the supporting body 238 of the articulation 206. Where the supporting body comprises two opposing tines 220,222, the insulating component may be located in between these two tines. More specifically, the insulating component 218 may be located between a first tine 220 and the end effector element 202 of the surgical instrument. The insulating component 218 may comprise a feature for housing the electrical connector 214. The housing of the electrical connector 214 by the insulating component 218 can be used to support the electrical connector against the electrical component 204. This therefore strengthens the electrical connection between the electrical connector 214 and the end effector 200.

Where the insulating component 218 is located within the supporting body 238, the insulating component may be configured to interfere with the supporting body such that rotation of the insulating component about the first axis 208 is limited when the end effector 200 rotates about the first axis. That is, the insulating component 218 may comprise at least one feature that is configured to interfere with a corresponding feature of the supporting body 238 when the end effector 200 rotates about the first axis 208. In one example, this feature of the insulating component 218 is a proximal surface 228 of the insulating component. The proximal surface 228 of the insulating component in this context is the surface of the insulating component that is closest to the shaft of the instrument. The corresponding feature of the supporting body 238 may be a distal surface 230 of the supporting body. The distal surface 230 of the supporting body is a surface of this component, with the exception of the tines, that is furthest from the shaft. When it is in the straight configuration, the proximal surface 228 of the insulating component may be separated from the distal surface 230 of the supporting body by a clearance distance d₁. The clearance distance d₁ may be such that, when the end effector 200 rotates about the first axis 208, the distance decreases such that rotation of the insulating component 238 is limited. More specifically, as the end effector element 202 rotates about the first axis 208, at least part of the proximal surface 228 of the insulating component abuts a corresponding part of the distal surface 230 of the supporting body. This abutment prevents any further rotation of the insulating component 218 relative to the supporting body 238. This in turn may limit rotation of the electrical connector 214 that is housed by the insulating component 218. The clearance distance may be between 0.05 and 2 mm. For example, the clearance distance may be 1 mm.

The electrical connector 214 may be electrically connected to the end effector 200 by a connecting means 232. The connecting means 232 provides the electrical connection between the electrical component 204 and the electrical connector 214. The connecting means 232 may also be configured to apply a compressive force to the electrical connector 214. The compressive force may constrain the electrical connector 214 against the insulating component 218. The constraining of the electrical component 204 against the insulating component ensures that a good electrical connection is maintained between the electrical component 204 and the electrical connector 214. In an alternative example, the surgical instrument may not comprise a connecting means. Instead, an electrical connection between the electrical component 204 and the electrical connector 214 is obtained from direct contact of the end effector 200 by the electrical connector 214.

The connecting means 232 may be configured to apply its compressive force by means of a spring mechanism. Thus, the connecting means may comprise a spring, which may be referred to as a “compression spring”. In the example illustrated in FIG. 3, the connecting means 232 comprises a disc spring. the connecting means may alternatively comprise a helical compression spring, a conical spring, or any other suitable type of spring. The connecting means 232 may comprise another type of component that is capable of applying a compressive force to the electrical connector whilst maintaining an electrical connection between the connector and the end effector. For example, the connecting means 232 may be a retaining ring such as a circlip. In order to provide a suitable electrical connection, the connecting means 232 may be made of any suitable electrically conducting material. For example, the connecting means 232 may be made of a metal, such as copper, iron, or steel. The connecting means 232 may be made of a material that is suitable for pharmaceutical use, such as stainless steel 316/316L.

The connecting means 232 may further comprise a securing mechanism configured to secure the means to the end effector 200. The securing mechanism is useful because it can ensure that the connecting means 232 does not move with respect to the end effector 200. Thus, it ensures that the sliding connection between the electrical cable 214 and the end effector 200 is provided only by the rotational freedom of the electrical connector 214 with respect to the end effector. In one example, the securing mechanism is a keyed joint. A keyed joint comprises a machine key that extends between two interfacing components, and a corresponding groove located in at least one of the two interfacing components within which the key is located. The location of the key within the at least one groove is used to secure the surfaces together. In the example illustrated in FIG. 3, the electrical component 204 of the end effector and the connecting means 232 may each comprise grooves within which a key can be located. Alternatively, only one of the connecting means 232 and the electrical component 204 may comprise a groove, with the key being integral with and protruding from the corresponding component. Examples of alternative securing mechanisms are the application of a filler material between the connecting means 232 and the electrical component 204 to provide a welded joint or the use of mechanical fasteners. In a further example, the securing mechanism could comprise an adhesive for connecting the connecting means 232 to the electrical component 204. Instead of comprising a distinct securing mechanism, the connecting means may form part of the end effector of the surgical instrument. That is, the end effector could have a deflectable or bendable feature that provides an electrical connection between the electrical component 204 and the electrical connector 214 whilst also applying a compressive force to the electrical connector.

The feature for housing the electrical connector 214 by the insulating component 218 may be a groove 234 within which the electrical connector 214 can be located. The groove 234 may be recessed from an outer surface 236 of the insulating component. The outer surface 236 of the insulating component is a surface that faces the end effector element 202 of the end effector. By recessing the groove 234 from this outer surface 236, the electrical connector 214 can be positioned so that it is facing the end effector element 202. As mentioned above, this configuration enables an electrical connection between the electrical connector 214 and the end effector 200 to be provided. In the example illustrated in FIG. 3, the electrical connector 214 is completely housed within the grove 234. However, in an alternative example, only part of the electrical connector 214 may be housed within the groove 234. The groove 234 may have a corresponding geometry to that of the electrical connector 214. That is, where the electrical connector is in the shape of a ring, the groove 234 may also have the shape of a ring. The inner and outer perimeters of the groove 234 may be slightly larger than those of the electrical connector 214, so that the electrical connector can fit within the groove. The use of a groove as a housing feature is advantageous as the electrical component 214 is set back from the end effector element 202. This shields the electrical connector 214 from the end effector element and reduces the likelihood of unintended burns and electrocution of the patient via charging of the end effector element 202.

An alternative arrangement for an improved electrosurgical instrument to be attached to a surgical robot is illustrated in FIG. 4. The electrosurgical instrument of FIG. 4 comprises many features in common with those of the instrument illustrated in FIG. 3. The instrument of FIG. 4 comprises a shaft and an end effector 300. The instrument further comprises an articulation 306 connecting the shaft to the end effector 300. The 306 articulation comprises a supporting body 338, that is connected to the shaft by a joint. The supporting body 338 may comprise a first tine 320 and a second tine 322. The first and second tine may be positioned and orientated as described above with respect to FIG. 3.

The surgical instrument of FIG. 4 differs from the corresponding instrument illustrated in FIG. 3 in that its end effector comprises a pair of end effector elements comprising a first end effector element 302a and a second end effector element 302b. The end effector 300 further comprises a first joint 310a permitting the first end effector element 302a to rotate about a first axis 308a. The first axis 308a may be transverse to the longitudinal axis 326 of the shaft. A second joint 310b permits the second end effector element 302b to rotate about a second axis 308b. The second axis 308b may also be transverse to the longitudinal axis 326 of the shaft. The second axis 308b may be parallel to the first axis 308a. The first axis 308a and the second axis 308b may be collinear. In one example the first and second axes are the same axis.

Each end effector element of the end effector 300 comprises an electrical component 304a, 304b corresponding to the electrical component 204 illustrated in FIG. 3. Each electrical component 304a, 304b is provided with electrical current by a corresponding electrical cable 312a, 312b and electrical connector 314a, 314b. The electrical cables 312a, 312b may be composed as is described with respect to FIG. 1 above. For example, the first end effector element 302a is electrically connected to a first electrical cable 312a by a first electrical connector 314a. The first electrical connector 314a provides a sliding electrical connection between the first electrical cable 312a and the electrical component 304a of the end effector. Thus, the first electrical connector 314a provides a sliding electrical connection between the first electrical cable 312a and the first end effector element 302a to which the first electrical component 304a is connected. The sliding electrical connection is such that the first end effector element 302a is permitted to rotate about the first axis 308 independently of the first electrical cable 312a whilst the electrical connection between the first electrical cable and the first end effector element is maintained. The first electrical connector 314a may be designed as described above with respect to the electrical connector 214 of FIG. 3. For example, the first electrical connector 314 may extend at least partially around a circumference surrounding the first axis 308.

Similarly, the second end effector element 302b is electrically connected to a second electrical cable 312b by a second electrical connector 314b. The second electrical connector 314b provides a sliding electrical connection between the second electrical cable 312b and the second electrical component 304b of the end effector. Thus, the second electrical connector 314b provides a sliding electrical connection between the second electrical cable 312b and the second end effector element 302b to which the second electrical component 304b is connected. The sliding electrical connection is such that the second end effector element 302b is permitted to rotate about the second axis independently of the second electrical cable 312b whilst the electrical connection between the second electrical cable and the second end effector element is maintained. The second electrical connector 314b and may also be designed as described above with respect to the electrical connector 214 of FIG. 3. For example, the second electrical connector 314b may extend at least partially around a circumference surrounding the second axis.

Each of the first and second electrical connectors may be connected to the end effector using a respective connecting means 332a, 332b. Each of the respective connecting means may further comprise a corresponding securing mechanism. The connecting and securing mechanism may be as described above with respect to FIG. 3. The surgical instrument of FIG. 4 may further comprise an insulating component 318 corresponding to insulating component 218 described above with respect to FIG. 3. The insulating component may comprise grooves 334a, 334b corresponding broadly to the groove 234 illustrated on insulating component 218. That is, the insulating component 318 may have a groove for each electrical connector 314a, 314b that is recessed from a respective outer surface of the insulating component. The insulating component may be located between the first end effector element 302a and the second end effector element 302b. Thus, a first groove 334a may be recessed from a first outer surface 336a that faces the first end effector element 302a. A second groove 334b may be recessed from a second outer surface 336b that faces the second end effector element 302b. As the first end effector element 302a opposes the second end effector element 302b, the first outer surface 336a is on an opposing side of the insulating component to the second outer surface 336b of the insulating component. The first electrical connector 314a is therefore electrically isolated from the second electrical connector 314b. This may be important as it ensures that electrical signals provided by the first cable 312a do not interfere with those provided by the second cable 312b. Interference between these electrical signals would cause the electrical components of the instrument to short circuit, which would prevent current/energy from passing through the tissue of the patient. Thus, by electrically isolating the first electrical connector from the second electrical connector, the electrosurgical capabilities of the end effector elements are optimised.

The example illustrated in FIG. 4 illustrates each end effector element 302a, 302b of the end effector 300 being electrically connected to a corresponding electrical cable 312a, 312b by a respective electrical connector 314a, 314b. Thus, the instrument illustrated in FIG. 4 is a bipolar surgical instrument. In an alternative example, the instrument of FIG. 4 may be a monopolar surgical instrument. In this example, the instrument may comprise a single electrical cable and a single electrical connector connecting the electrical cable to one of the two end effector elements of the instrument. The instrument also comprises a pair of external insulators 324a, 324b configured to insulate the electrically charged components of the instrument such as the electrical connectors and the electrical components from other parts of the instrument and from the patient. In FIG. 4 each external insulator 324a, 324b is connected to a corresponding electrical component 304a, 304b. In an alternative example, the first and second electrical components may be insulated by a common external insulator.

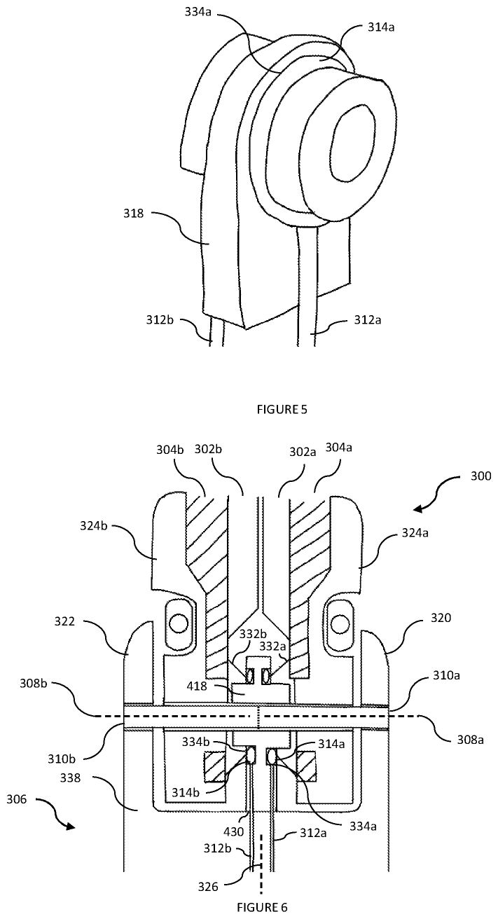

An alternative view of the insulating component 318 of FIG. 4 is illustrated in FIG. 5. FIG. 5 provides an isometric view of the insulating component, in which the geometry of the first electrical connector 314a can be seen. The electrical connector 314a may be housed within a groove 334a of the insulating component. In FIG. 5, the electrical cables 312a, 312b are illustrated as extending parallel to but externally of the insulating component 318. In an alternative example, the electrical cables 312a, 312b extend through (i.e., internally to) the insulating component before terminating at the electrical connectors 314a, 314b. Similarly, electrical cables 312a, 312b may extend along the articulation of the instrument either externally (as illustrated in FIG. 4) or internally to the articulation. The same options apply to the instrument arrangement illustrated in FIG. 3.

A first alternative configuration of the distal portion of the electrosurgical instrument to be attached to a surgical robot illustrated in FIG. 4 is illustrated in FIG. 6. The instrument of FIG. 6 is substantially the same as the corresponding instrument in FIG. 4, as represented by the use of corresponding reference numerals in FIG. 6 to those used in FIG. 4. The sole distinction of the instrument illustrated in FIG. 6 over the instrument described in FIG. 4 is that, instead of being separated from the supporting body by a clearance distance d₁, the insulating component 418 is secured at its proximal end 430 to the supporting body. The insulating component 418 may be secured to the supporting body 338 using any suitable adhering mechanism, such as the use of an adhesive or mechanical fasteners. The securing of the insulating component 418 to the supporting body 338 provides an alternative mechanism for ensuring that rotation of the insulating component is limited when the end effector elements are articulated about the first and/or second axes. This, in turn, reduces the likelihood of rotation of the electrical connectors 314a, 314b, and therefore the likelihood of the electrical cables 312a, 312b being subjected to excessive stress. The securing of an insulating component at a proximal end 430 to the supporting body 338 can additionally be incorporated into the instrument illustrated in FIG. 3.

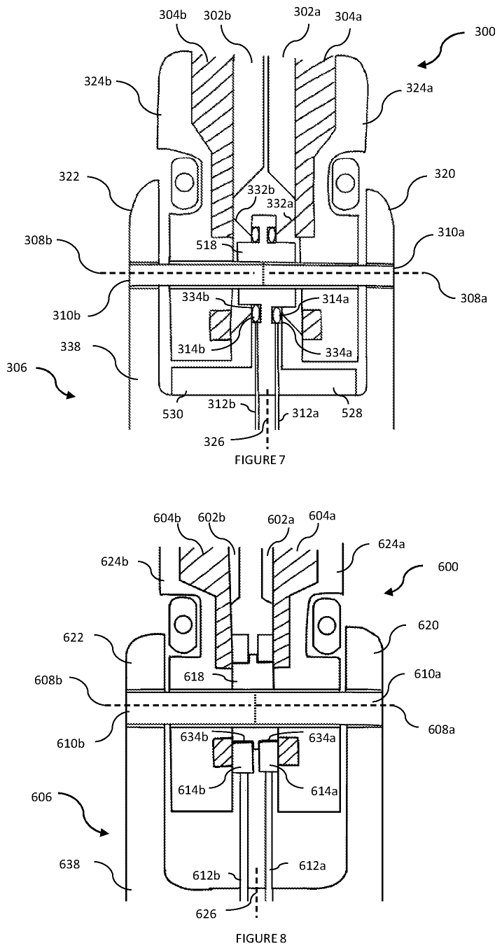

A second configuration of the distal portion of the electrosurgical instrument of FIG. 4, to be attached to a surgical robot, is illustrated in FIG. 7. The instrument in FIG. 7 is substantially the same as the corresponding instruments in FIGS. 4 and 6, as represented by the use of corresponding reference numerals in FIG. 7 to those used in those figures. The sole distinction between the instrument illustrated in FIG. 7 and those of FIGS. 4 and 6 is that the insulating component 518 comprises extruded portions 528, 530 that extend along the distal surface of the supporting body 338. A first extruded portion 528 extends away from the centre of the insulating component 518 towards the first tine 320 of the supporting body. A second extruded portion 530 extends away from the centre of the insulating component 518 towards the second tine 322 of the supporting body. In FIG. 6, the extruded portions are integral to the insulating component 318. The extruded portions may be symmetrical. The purpose of the extruded portions is to increase the creepage distance between the electrical connectors 314a, 314b and the supporting body 338. Creepage distance is defined as the shortest distance along the surface of a solid insulating material between two conductive parts. By increasing the creepage distance, the electrical isolation provided by the insulating component can be optimised. In an alternative example, first and second extruded portions 528, 530 may be provided by a second insulating component that is auxiliary to the main insulating component 518.

A further configuration of the distal portion of the surgical instrument in FIGS. 4, 6 and 7, to be attached to a surgical robot, is illustrated in FIG. 8. As with the arrangements illustrated in the preceding figures, the surgical instrument of FIG. 8 comprises a first end effector element 602a and a second end effector element 602b, each end effector element comprising a corresponding electrical component 604a, 604b. The instrument further comprises an articulation 606 with a supporting body 638. The instrument comprises first and second joints 610a, 610b, permitting the end effector elements to rotate about respective first and second axes 608a, 608b. The supporting body 638 comprises a first time 620 and a second tine 622. The instrument further may comprise external insulators (not shown) for insulating the electrical components of the end effectors elements from other metal parts of the surgical instrument and from the patient. The instrument also comprises first and second electrical cables 612a, 612b that are electrically connected to respective electrical components of the end effector by electrical connectors 614a, 614b. The electrical cables and electrical connectors illustrated in FIG. 8 are the same as those described above with respect to FIGS. 3 and 4.

As with the surgical instruments illustrated in FIGS. 3 to 7, the instrument of FIG. 8 comprises an insulating component 618. The insulating component 618 may extend cylindrically around the first and second joints 608a, 608b. The insulating component 618 may be a locking ring. The insulating component 618 may be two locking rings. The insulating component 618 is configured to be secured to the end effector. That is, the insulating component 618 may comprise at least one securing mechanism to secure it to the end effector. More specifically, the at least one securing mechanism may secure the insulating component 618 to an end effector element 602a, 602b of the end effector. Where the insulating component 618 comprises two locking rings, a first locking ring may be secured to the first end effector element 602a by a first securing mechanism. Similarly a second locking ring may be secured to the second end effector element 602b by a second securing mechanism. An example of a suitable securing mechanism is a threaded male component extruded from a locking ring that is configured to screw into a corresponding tapped female hole in a respective end effector element. This mechanism allows the locking ring to be screwed and unscrewed from its respective end effector element.

The insulating component 618 is configured to house the electrical connectors 614a, 614b. More specifically, the electrical connectors 614a, 614b are configured to extend at least partially around the circumference of the insulating component 618. The insulating component 618 may comprise a cylindrical outer surface 630. The insulating component may further comprise first and second grooves 634a, 334b. Each groove 634a, 334b is recessed from the cylindrical outer surface 630 of the insulating component. Each groove is configured to house at least part of a respective electrical connector 614a, 614b. The insulating component is located between the first end effector element 602a and the second end effector element 602b. Thus, the first groove 634a may be recessed from the outer surface at a first side of the insulating component adjacent to the first end effector element 602a. The second groove 634b may be recessed from the outer surface at a second side of the insulating component adjacent to the second end effector element 602b. As the first end effector element 602a opposes the second end effector element 602b, the first side of the insulating component opposes the second side of the insulating component. The first electrical connector 614a is therefore electrically isolated from the second electrical connector 614b when the connectors are housed within their respective grooves. This is important as it ensures that electrical signals provided by the first cable 612a do not interfere with those provided by the second cable 612b. Thus, the electrosurgical capabilities of the end effector elements are optimised. The function of the groove(s) of the insulating component 618 is the same as that of the corresponding grooves of the insulating component 318 illustrated in FIG. 4. The locking ring may alternatively comprise a different type of securing mechanism, such as a retaining ring that can adjoin a corresponding electrical connector 614a, 614b around the circumference of the locking ring at a surface of the connector that opposes the end effector element.

As with the instrument illustrated in FIG. 4, each end effector element 602a, 602b of the end effector 600 in FIG. 7 is electrically connected to a corresponding electrical cable 612a, 612b by a respective electrical connector 614a, 614b. Thus, the instrument illustrated in FIG. 7 is a bipolar surgical instrument. In an alternative example, the instrument of FIG. 7 may be a monopolar surgical instrument. In this example, the instrument may comprise a single electrical cable and a single electrical connector connecting the electrical cable to one of the two end effector elements of the instrument. Thus, the insulating component 618 may only comprise a single groove for housing that single electrical connector. Similarly, the instrument may comprise a single connecting means and for the single electrical connector. The insulating component 618 with a single groove may be applied to the instrument illustrated in FIG. 3.

For each of the surgical instruments illustrated in FIGS. 3 to 8, the electrical connector(s) may require additional means to ensure that they are not fixed to the end effector of the instrument when the effector element is actuated about a respective joint. To further ensure that a sliding electrical connection is maintained, an outer surface of the electrical connector(s) may be provided with an anti-friction coating. The electrical connector(s) may be treated to provide this coating. For example, the outer surface of each electrical connector may be coated in an anti-friction material, or a lubricant. Alternatively, the outer surface of each electrical connector may be provided with a surface finish that reduces friction. For example, the surfaces may be polished to provide an anti-friction coating. In particular, at least the outer surface of the electrical connector that faces its respective end effector element is coated. This is the surface of the electrical connector that is arranged to provide a sliding electrical connection. Additionally, an outer surface of the electrical connector that interfaces with the insulating component may be coated in an anti-friction material. This outer surface may be the same surface as the outer surface that faces the end effector element. The anti-friction material may be any suitable lubricating product that is capable of reducing friction. The anti-friction material may be a dry-film lubricant. The frictional force between the electrical connector and the end effector may alternatively or additionally be reduced by reducing the area of contact between these two components (i.e., by decreasing the size of the connector), or by reducing the spring force of the electrical connector (e.g., by using a different material).

The end effector elements of the surgical instruments illustrated in FIGS. 3 to 8 may be any suitable end effector elements that are capable of performing a surgical procedure. In the examples illustrated in FIGS. 4 to 8, the first and second end effector elements may be a pair of opposing blades. For example, the first and second end effector elements may be a pair of scissors. Alternatively, the first and second end effector elements may be a pair of smooth jaws, serrated jaws, a pair of shears, a pair of blades of a gripping tool or any other suitable electrosurgical end effector. In the example illustrated in FIG. 3, the end effector element may be a knife, a cauteriser, or any other suitable electrosurgical end effector.

Similarly to the instrument described in FIGS. 2A and 2B, the instrument of any of FIGS. 3 to 8 may either be a monopolar instrument or a bipolar instrument. That is, each of the illustrated instruments may comprise either a single electrical component or a pair of electrical components. Where the surgical instrument is a monopolar surgical instrument, the instrument may comprise a single electrical cable and a single electrical connectors to provide electrical current to the single electrical component. Where the surgical instrument is a bipolar surgical instrument, the instrument may comprise a pair of electrical cables and a pair of electrical connectors to transfer electrical current.

In one example, the at least one electrical component of the surgical instrument is an emitter. The emitter is configured to generate radio frequency (RF) waves that are transferred to a part of the end effector element that is configured to contact the body of a patient. In an alternative example, the at least one electrical component is another type of emitter of energy waves. The energy waves emitted by the emitter may be high frequency energy waves. For example, the electrical component may be an emitter of radio frequency (RF) waves, microwaves, infrared waves, ultrasound waves or waves of any other suitable frequency that can be used to heat surgical tissue. The type of wave emitted is dependent on the frequency of current that is provided to the end effector. The sliding electrical connector disclosed herein is particularly advantageous for use with electrosurgical instruments that generate high frequency energy waves, such as microwaves or ultrasound waves. This is because such instruments require heavily insulated cables such as coaxial cables to efficiently transmit high frequency electrical current, and such cables are stiff and more prone to stress when bending. Thus, an arrangement that reduces bending of electrical cables, such as those described herein, is advantageous.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A robotic surgical instrument configured to perform a surgical procedure, the instrument comprising:
a shaft;
an end effector comprising an electrical component;
an articulation connecting the shaft to the end effector, the articulation comprising a supporting body connected to the end effector by a first joint and to the shaft by a second joint, the first joint permitting the end effector to rotate relative to the shaft about a first axis that is transverse to the longitudinal axis of the shaft;
an electrical cable extending along the shaft and being configured to provide electrical current to the electrical component of the end effector;
an electrical connector extending at least partially around a circumference surrounding the first axis, the electrical connector providing a sliding electrical connection between the electrical cable and the end effector, such that the end effector is permitted to rotate about the first axis independently of the electrical cable whilst the electrical connection between the electrical cable and the end effector is maintained; and
an insulating component configured to house the electrical connector within the surgical instrument, the insulating component comprising an outer surface that faces an end effector element of the end effector.

2. The robotic surgical instrument of claim 1, wherein the electrical connector extends around the entirety of the circumference surrounding the first axis.

3. The robotic surgical instrument of claim 1, wherein the electrical connector is in the shape of a ring.

4. The robotic surgical instrument of claim 1, wherein the electrical cable is connected to the electrical connector at a section of the electrical connector that is closest to the shaft.

5. The robotic surgical instrument of claim 1, wherein the electrical connector is integral with the electrical cable.

6. The robotic surgical instrument of claim 1, wherein the insulating component is configured to interfere with the supporting body such that rotation of the insulating component about the first axis is limited when the end effector rotates about the first axis.

7. The robotic surgical instrument of claim 1, wherein the insulating component is secured at a proximal end to the supporting body.

8. The robotic surgical instrument of claim 1, wherein the electrical connector is electrically connected to the end effector by a connection which is configured to apply a compressive force to the electrical connector to constrain the electrically connected against the insulating component.

9. (canceled)

10. The robotic surgical instrument of claim 1, wherein the electrical connector is electrically connected to the end effector by a disc spring.

11. The robotic surgical instrument of claim 1, wherein the electrical connector is electrically connected to the end effector by a securing mechanism configured to secure the securing mechanism to the end effector.

12. The robotic surgical instrument of claim 11, wherein the securing mechanism is a keyed joint.

13. The robotic surgical instrument of claim 1, wherein the insulating component comprises a groove that is recessed from the outer surface and configured to house at least part of the electrical connector.

14. The robotic surgical instrument of claim 1, wherein the insulating component is a locking ring configured to be secured to the end effector, and the electrical connector is configured to extend at least partially around the circumference of the locking ring.

15. The robotic surgical instrument of claim 14, wherein the insulating component comprises a cylindrical outer surface and a groove that is recessed from the outer surface and configured to house at least part of the electrical connector.

16. The robotic surgical instrument of claim 1, wherein the electrical connector comprises an outer surface that is arranged to provide the sliding electrical connection and that is provided with an anti-friction coating.

17. The robotic surgical instrument of claim 1, wherein the instrument is a monopolar surgical instrument or a bipolar surgical instrument.

18. The robotic surgical instrument of claim 1, wherein the electrical cable is a first electrical cable, and the electrical connector is a first electrical connector;
the end effector comprises a first end effector element configured to rotate about the first axis and a second end effector element configured to rotate about a second axis;
the first electrical connector provides a sliding electrical connection between the first electrical cable and the first end effector element of the end effector; and
the robotic surgical instrument further comprises a second electrical cable and a second electrical connector, the second electrical connector providing a sliding electrical connection between the second electrical cable and the second end effector element of the end effector, such that the second end effector element is permitted to rotate about the second axis independently of the second electrical cable whilst the electrical connection between the second electrical cable and the second end effector element is maintained.

19. The robotic surgical instrument of claim 18 wherein the second electrical connector extends at least partially round a circumference surrounding the second axis.

20. The robotic surgical instrument of claim 18, wherein the first axis and the second axis are collinear

21. The robotic surgical instrument of claim 18, wherein the insulating component is located between the first end effector element and the second end effector element such that the first electrical connector is electrically isolated from the second electrical connector, and the insulating component is configured to interfere with the supporting body such that rotation of the insulating component about the first axis is limited when the end effector rotates about the first axis.

22.-26. (canceled)
